(19) United States
(12) Patent Application Publication
(10) Pub. No.: US 2005/0019400 A1
(43) Pub. Date: Jan. 27, 2005

(54) Controlled-release drug delivery system

(76) Inventors: Thomas William Deveney, Browns Mills, NJ (US); David R. Friend, Princeton, NJ (US); George Robert Pawlo, Long Valley, NJ (US); Kerrie L. Ziegler, Hammonton, NJ (US)

Correspondence Address: DANN, DORFMAN, HERRELL & SKILLMAN, P.C., SARNOFF CORPORATION, 1601 MARKET STREET, SUITE 720, PHILADELPHIA, PA, 19103, US

(21) Appl. No.: 10/764,786

(22) Filed: Jan. 26, 2004

Related U.S. Application Data
(60) Provisional application No. 60/442,859, filed on Jan. 27, 2003.

(51) Int. Cl. A61K 9/22

(52) U.S. Cl. 424/468; 604/890.1

(57) ABSTRACT

A controlled-release drug delivery system advantageously includes an open-ended, inflexible sleeve, at least two controlled-release layers and two open-center caps. Each controlled-release layer abuts a sealing surface that is located within and near each end of the sleeve. The caps seal each controlled-release layer against the abutting sealing surface. One or more dose units of drug are disposed in a region that is formed between the controlled-release layers. The controlled-release layers dissolve, at a predetermined rate, by the action of body fluids that are in contact with those layers through the center of the caps. Release of drug is delayed at least until the controlled-release layers dissolve. The dose unit itself, which is advantageously a core, can be tailored to provide an extended period of drug release. One or more dose units that provide an immediate release component can also be disposed near each end of the sleeve.

### FIELD OF THE INVENTION

The present invention relates to controlled-release drug delivery systems and dosage forms.

### BACKGROUND OF THE INVENTION

Controlled-release drug delivery systems (also referred to herein as “controlled-release drug dosage forms”) are capable of releasing a drug in an animal (e.g., a human, livestock, etc.) in accordance with one or more pre-selected conditions. The pre-selected condition can be, for example, a time delay, wherein drug release is delayed for some time after the dosage form has been administered. Another pre-selected condition is a time period, wherein drug is released from the dosage form over an extended period of time.

Often, the ideal release profile for a controlled-release dosage form is “zero order.” A zero-order drug release profile means that the drug delivery is independent of time (at least over a certain time period). This ideal is, however, difficult to achieve. The difficulty lies in the fact that for most controlled-release dosage forms, as the drug level inside the dosage form decreases, the rate of drug release also decreases. Consequently, controlled-release dosage forms often show two distinct phases of drug release: an initial phase, which might or might not be linear, and a second phase that is not linear, reflecting the rapid depletion of the drug from the dosage form.

In an attempt to at least approximate a zero-order release profile, some controlled-release dosage forms deliver drug as a series of small doses. In some systems, a “mechanical” release mechanism is used to deliver doses of drug from an open-ended housing. The release mechanism is typically a fluid-activated driving element, a physical implementation of which is a plug of material that swells upon contact with fluid. The release mechanism, which is disposed at one end of the housing, activates by imbibing liquid through a permeable region of the otherwise impermeable housing. As the material swells, it pushes the dosage units through the housing toward the open end. Each dose unit is sequentially released through the open end. Controlled-release drug dosage forms of this type are disclosed in U.S. Pat. Nos. 4,723,958, 5,017,381, and 5,938,654.

In some other controlled-release dosage forms, the release mechanism is a diffusion process. In some diffusion-based dosage forms, a drug that is embedded in a matrix of an insoluble substance gradually diffuses into the ambient environment. Controlled-release dosage forms of this type are disclosed in patent publications WO 89/09066, WO 91/04015, WO 95/22962, and WO 99/51208.

In assignee's own U.S. Pat. application Ser. No. 09/766,695, a multi-step drug dosage form was described in which dissolution serves as the primary drug-release mechanism. The dosage form advantageously includes an impermeable structural form that contains a stack of alternating separators and dose units. The structural form is open at one end, but the open end is blocked and sealed by a separator. That separator dissolves (at a pre-determined rate) to expose a dose unit(s) that is behind it. The exposed dose unit(s) dissolves into bodily fluids. Meanwhile, the next separator begins dissolving (at the same or different pre-determined rate) to eventually expose a dose unit behind it, and so forth. This sequential dissolving of separators to expose successive dose units provides a pulsatile or sustained-release profile.

The controlled-release dosage forms and systems described above have drawbacks. In some cases, the drawbacks are uniquely associated with dosage-form type (e.g., mechanical release, diffusion, etc.). More generally though, most of the controlled-release dosage forms are subject to structural integrity problems and would benefit from improvements in manufacturing and structural design.

### SUMMARY OF THE INVENTION

The present invention is a controlled-release drug delivery system that avoids some of the drawbacks of the prior art.

In accordance with the illustrative embodiment, the drug delivery system includes a sleeve, at least two controlled-release layers, and two caps. The sleeve, which is open at both ends, is inflexible and impermeable to body fluids for the duration of controlled drug release. In some embodiments, the controlled-release layers dissolve at a desired rate on exposure to body fluids. The dissolution rate is controlled primarily by layer thickness, layer composition, or both. The caps are ring-like, with open central regions.

The controlled-release layers are received within the sleeve. Each layer seals against a sealing surface located near each end of the sleeve. In the illustrative embodiment, the sealing surface is implemented as a ledge or shoulder, which encircles the inner surface of the sleeve near each end thereof.

The caps are also received within each sleeve. A marginal region of each cap abuts a marginal region of a respective controlled-release layer, such that each controlled-release layer is sandwiched between a respective sealing surface and the cap. The caps are fixed within the sleeve, advantageously by a friction fit. Since the sleeve and caps are inflexible, this arrangement creates a positive seal and provides a fluid-tight region that is bounded at either end by the controlled-release layers.

One or more dose units, which can be implemented in any of a variety of dosage forms (e.g., tablet, caplet, capsule, core, loose powder, etc.), are disposed within the fluid-tight region. Until such time as the controlled-release layers dissolve, these dose units remain isolated (in the fluid-tight region) from the ambient environment of the gastrointestinal tract. One or more dose units can also be disposed near each end of the sleeve in the open central region of a respective cap. Since the latter dose units are outside of the liquid-tight region, they are exposed to the ambient environment and are available for immediate drug release.

In some other embodiments, the controlled-release layers do not dissolve. They are, however, permeable to body fluids. Body fluids diffuse through the controlled-release layers, dissolve the dose unit(s), and diffuse back across the controlled-release layer, to deliver drug to an animal's system. By regulating the rate of diffusion across the controlled-release layers, a sustained drug release profile can be obtained.

Thus, in accordance with the illustrative embodiment, release of drug from these dose units can be delayed (for some period of time after administration), sustained (over an extended period of time), or both. The controlled-release layers, which:
dissolve at a rate that provides the desired delay; or
do not dissolve but, rather, control the rate of diffusion of drug;
or both,
   are the primary mechanism for providing delayed release and sustained release of drug.

### BRIEF DESCRIPTION OF THE DRAWINGS

FIG. 1 depicts a cross-sectional view of a controlled-release drug delivery system in accordance with the illustrative embodiment of the present invention.

FIG. 2 depicts an exploded view of the controlled-release drug delivery system of FIG. 1.

FIG. 3 depicts a cross-sectional view of the system of FIG. 1, showing detail of the interface between the cap, sleeve and controlled-release layer.

FIG. 4 depicts the controlled-release drug delivery system of FIG. 1 including dose units, wherein the dosage form of the dose units is a tablet.

FIG. 5 depicts the controlled-release drug delivery system of FIG. 1 including dose units, wherein the dosage form of the dose units is a core.

FIG. 6 depicts a delayed release profile of active ingredient that is delivered from a drug delivery system in accordance with the illustrative embodiment of the present invention.

FIG. 7 depicts a sustained release profile of active ingredient, which is in the form of a tablet, and which is delivered from a drug delivery system in accordance with the illustrative embodiment of the present invention.

FIG. 8 depicts a sustained release profile of active ingredient, which is in the form of a core, and which is delivered into intestinal fluid from a drug delivery system in accordance with the illustrative embodiment of the present invention.

FIG. 9 depicts a sustained release profile of active ingredient, which is in the form of a core, and which is delivered into gastric fluid from a drug delivery system in accordance with the illustrative embodiment of the present invention.

FIG. 10 depicts a release profile of an active ingredient that is delivered from a drug delivery system in accordance with the illustrative embodiment of the present invention, wherein the release profile is characterized by a period of immediate release, then a period of delay, and then a period of sustained release.

FIG. 11 depicts release profiles of two variations of a drug delivery system in accordance with the illustrative embodiment of the present invention, wherein composition of the controlled-release layers are varied.

FIG. 12 depicts release profiles of two variations of a drug delivery system in accordance with the illustrative embodiment of the present invention, wherein thickness of the controlled-release layers are varied.

FIG. 13 depicts release profiles into acidic and basic mediums for a first variation of a drug delivery system in accordance with the illustrative embodiment of the present invention, wherein the dose unit has a first excipient composition.

FIG. 14 depicts release profiles into acidic and basic mediums for a second variation of a drug delivery system in accordance with the illustrative embodiment of the present invention, wherein the dose unit has a second excipient composition.

FIG. 15 depicts release profiles of two variations of a drug delivery system in accordance with the illustrative embodiment of the present invention, wherein one variation has active ingredient in the form of a core and the other variation includes active ingredient in the form of loose powder.

### DETAILED DESCRIPTION

The terms listed below are given the following definitions for use in this specification. Additional definitions are provided later in this Detailed Description.

The terms “active agent”, “pharmaceutical” and “drug” are used interchangeably herein and are defined as a compound, composition of matter, or mixture thereof that can be delivered from the drug-delivery system to produce a beneficial or useful result, such as the mitigation, diagnosis, cure, treatment, or prevention of a disease. This includes, in particular, any physiologically- or pharmacologically-active substance that produces a localized or systemic effect in animals. This also includes diagnostic and prophylactic agents.

The term “controlled release” means release of drug from a dosage form in a pre-determined manner or according to a pre-determined condition.

The term “delayed release” means release of drug at a time later than immediately after administration.

The term “deposit” means a single dose unit of drug held on a substrate.

The terms “deposition film,” “deposition substrate” and “substrate” are used interchangeably herein and means a material upon which a dose unit is disposed in forming a deposit.

The term “dissolve” means true dissolution, enzymatic degradation, bacterial digestion, erosion, and any other form of material breakdown.

The term “dosage amount” means an amount of drug needed to achieve a desired beneficial or useful effect.

The term “dosage form” means a formulation of a drug or drugs in a form administrable to an animal, wherein the term “animal” is intended to encompass a human. While the illustrative embodiment of the invention has been described primarily as being directed to oral dosage forms such as tablets, cores, capsules, caplets and loose powder, it is also applicable to dosage forms intended for other types of administration, such as, for example, vaginal and rectal suppositories, and implants.

The term “dose unit” means an isolated quantity of drug. In some embodiments, a dose unit includes a dosage amount of the drug; in other embodiments, a dose unit includes more or less than a dosage amount.

The term “extended release” or “sustained release” means release of drug from a dosage form over an extended period of time. Extended-release dosage forms enable a reduction in dosing frequency compared to immediate-release dosage forms.

The term “hydrophobic drug” means a drug that ranges from “sparingly soluble” to “practically insoluble or insoluble,” as follows:

| | Parts of Solvent Required |
|---|---|
| Descriptive Term | for 1 Part of Solute |
| Sparingly soluble | from 30 to 100 |
| Slightly soluble | from 100 to 1000 |
| Very slightly soluble | from 1000 to 10,000 |
| Practically insoluble, or insoluble | 10,000 and over |

The term “immediate release” means release of drug from a dosage form in a relatively brief period of time after administration, generally up to about 60 minutes.

The term “modified release” includes delayed release, extended release, and pulsed release.

The term “pharmaceutically acceptable” means that a drug, etc., can be introduced safely into an animal body (e.g., taken orally and digested, etc.).

The term “pulsed release” means a series of releases of drug.

The term “release mechanism” means a process by which drug is released from the dosage form.

The term “surfactant” means a surface active agent that displays wetting, detergent or soap-like qualities as those agents are understood by those skilled in the art. The term “surfactant” therefore includes ionic and nonionic surfactants or wetting agents commonly used in the formulation of pharmaceuticals, such as ethoxylated castor oil, benzalkonium chloride, polyglycolyzed glycerides, acetylated monoglycerides, sorbitan fatty acid esters, poloxamers, polyoxyethylene fatty acid esters, polyoxyethylene derivatives, monog lycerides or ethoxylated derivatives thereof, diglycerides or polyoxyethylene derivatives thereof, sodium docusate, sodium lauryl sulfate, magnesium lauryl sulfate, triethanolamine, cetrimide, sucrose laurate and other sucrose esters, glucose (dextrose) esters, simethicone, ocoxynol, dioctyl sodium sulfosuccinate, polyglycolyzed glycerides, sodium dodecylbenzene sulfonate, dialkyl sodium sulfosuccinate, fatty alcohols such as lauryl, cetyl and streryl, glycerylesters.

The illustrative embodiment of the present invention is a controlled-release drug delivery system. The system is typically (although not necessarily) orally administered. In some embodiments, a controlled-release drug delivery system in accordance with the illustrative embodiment is capable of delaying the release of drug for a predetermined amount of time after administration. This can be useful, for example, in conjunction with the administration of sleep-aids or for the administration of drugs for indications that are more prevalent in the morning, such as asthma and heart attacks.

In some other embodiments, the controlled-release drug delivery system is designed to release drug for an initial period of time, followed by a period of time in which drug is not released. After the period for delay has elapsed, the system can immediately deliver a dosage amount of drug, or, alternatively, release the dosage amount over an extended period of time.

This section now continues with a description of the structure of a controlled-release drug delivery system.

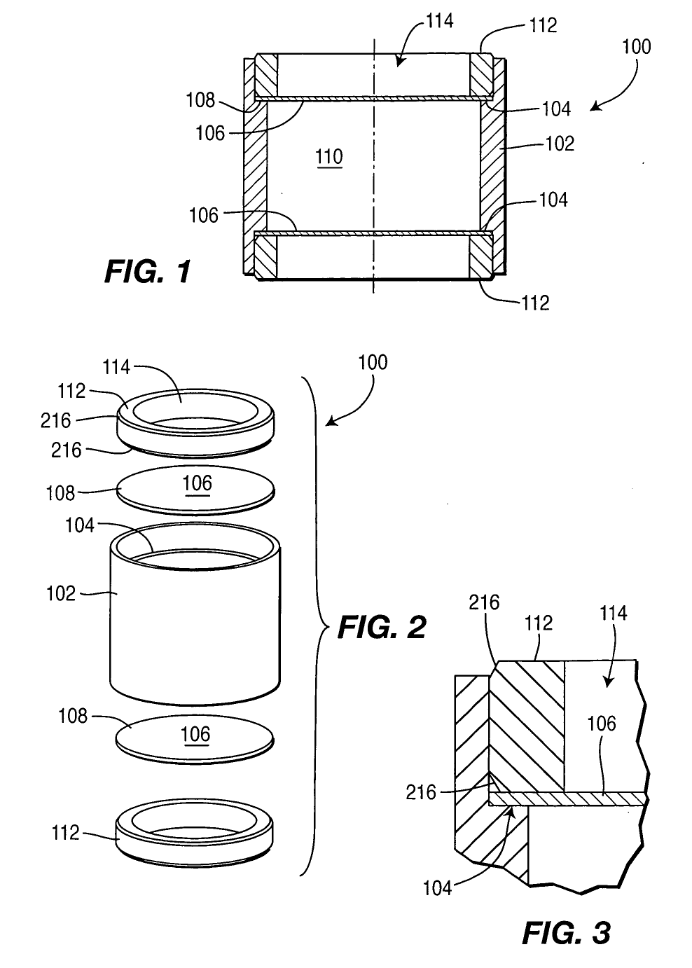

FIGS. 1 and 2 depict controlled-release drug delivery system 100 in accordance with the illustrative embodiment of the present invention. FIG. 1 depicts system 100 via a cross-sectional view and FIG. 2 depicts system 100 via an exploded, perspective view. As can be seen in those Figures, system 100 includes sleeve 102, controlled-release layers 106, and caps 112. These elements cooperate physically as shown.

Sleeve 100 is inflexible, impermeable to body fluids during the intended period for drug release, and is advantageously (but not necessarily) cylindrical in shape. Sleeve 102 is hollow; that is, it is open through to each end thereof. This inner, hollow portion of sleeve 102 is referred to herein as the “lumen.”

Near each end of sleeve 102 is a sealing surface, which is used to create a liquid-tight region within sleeve 102 for receiving dose units of drug. In the illustrative embodiment, the sealing surface is implemented as ledge, ridge or shoulder 104. (Only the “upper ledge” of sleeve 102 is depicted in FIG. 2.) In the illustrative embodiment, ledges 104 are created by enlarging the inside diameter of sleeve 102 near both ends. The transition between each larger-diameter region (near each end) and the (central) smaller-diameter region forms ledges 104.

Controlled-release layers 106 are configured to seal against ledges 104. Consequently, controlled-release layers 106 have a shape that at least approximates the cross section of the lumen of sleeve 102. For example, since the cross section of the lumen is circular in the illustrative embodiment, controlled-release layer 106 is advantageously configured as a disk. Furthermore, the diameter of each controlled-release layer 106 must be large enough to overlap ledge 104, yet small enough to fit within the lumen of sleeve 102.

Caps 112 are physically adapted to seal against controlled-release layers 106 and fix those layers in place against ledges 104. Caps 112 are configured as a “ring;” that is, they have a central, open region 114. Caps 112 are received by the lumen of sleeve 102 and advantageously engage the inside walls of sleeve 102 in a friction fit. By virtue of open region 114, controlled-release layers are exposed to the ambient environment (e.g., the gastrointestinal system, etc.) even when caps 112 are engaged to sleeve 102.

The outer diameter of caps 112 nearly match the inside diameter of sleeve 102 (near each end) to create a friction fit. As a consequence, it is advantageous to chamfer caps 112 to form sloping edges 216. The edge makes it easier to insert cap 112 into an end of sleeve 102. Alternatively, the edge of sleeve 102 can be chamfered, or both caps 112 and sleeve 102 can be chamfered.

FIG. 3 depicts a partial view of device 100 showing the region near ledge 104. FIG. 3 illustrates the manner in which a liquid-tight seal is formed as controlled-release layer 106 is pressed against ledge 104 by cap 112. Since sleeve 102 is inflexible, the seal created at ledge 104 by controlled-release layer 106 and cap 112 remains liquid-tight regardless of physical stresses that might otherwise cause flexing or distortion of a flexible sleeve. Furthermore, cap 112 is advantageously formed from the same material as sleeve 102 to prevent gapping, as might otherwise result during thermal cycling of dissimilar materials.

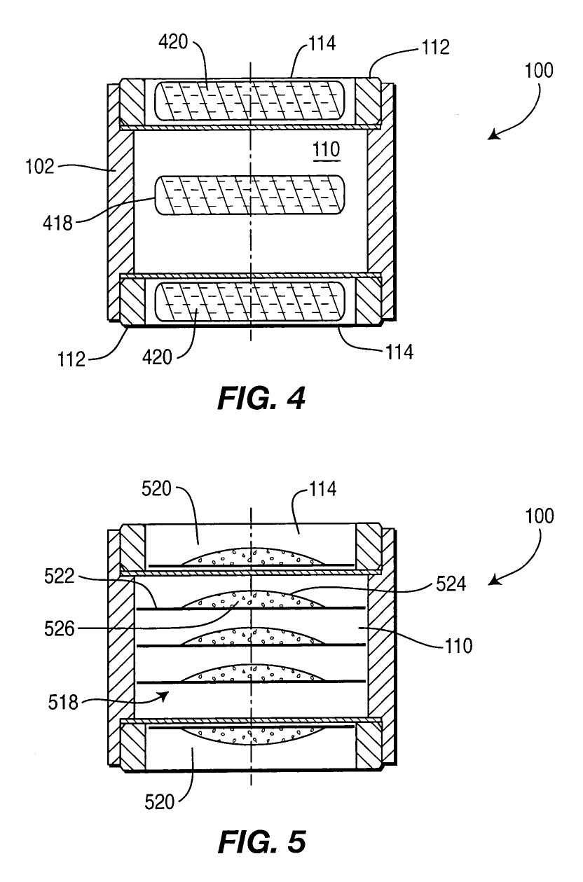

With reference to FIG. 1, the two controlled-release layers 106 are set-off from one another by ledges 104. Region 110 (between controlled-release layers 106) receives one or more dose units of drug (not depicted in FIG. 1, see FIGS. 4 and 5).

As previously described, sleeve 102 is impermeable to body fluids. Furthermore, the liquid-tight seal that is formed by ledge 104, controlled-release layer 106, and cap 112 is robust. As described in further detail later in this specification, in some embodiments, controlled-release layers 106 dissolve (at a predetermined rate that provides a desired delay for the release of drug). In some other embodiments, layers 106 do not dissolve. Rather, they serve as a diffusion barrier to provide a sustained release of drug.

In embodiments in which controlled-release layers 106 are impermeable, dose units of drug that are contained within region 110 are isolated from the ambient environment (e.g., the gastrointestinal tract of an animal) as long as controlled-release layers 106 remain intact. And they remain intact until they dissolve due to exposure to body fluids.

As controlled-release layers 106 dissolve, the one or more dosage forms that are contained within region 110 are exposed to the ambient environment (e.g., body fluids, etc.). Drug is then potentially available for dissolution into body fluids. In this manner, the rate of dissolution of controlled-release layers 106 is a key mechanism in creating delayed drug release from controlled-release drug delivery system 100. After layers 106 dissolve, the availability of drug depends upon certain characteristics of the dosage form of the unit dose(s).

As mentioned above, in some embodiments, controlled-release layers 106 do not dissolve; rather, they serve as diffusion barriers. In these embodiments, drug from the one or more dosage forms that are contained within region 110 is released to the ambient environment over a period of time, as limited by the rate of diffusion across controlled-release layers 106.

In some further embodiments, controlled-release layers 106 delay the release of drug as well as providing a sustained release.

In addition to the dose unit(s) of drug within region 110, dose unit(s) of drug can also be disposed in region 114 on the side of controlled-release layer 106 that is exposed to the ambient environment. Since dose units at this location are not isolated from the environment by controlled-release layers 106, they can serve as an immediate-release component of system 100.

FIGS. 4 and 5 depict, via cross-sectional view, controlled-release drug delivery system 100 including several dose units of drug. In particular, system 100 shown in FIG. 4 includes dose units 418 and 420, and system 100 depicted in FIG. 5 incorporates dose units 518 and 520. Dose units 420 and 520 are available for immediate release, while dose units 418 and 518 are subjected to controlled delivery since they are between controlled release layers 106 in region 110.

With reference to FIG. 4, dose units 418 and 420 are in tablet form. In the illustrative embodiment, one tablet 420 is depicted in region 114 at both ends of system 100. Furthermore, one tablet is disposed in region 110 between controlled-release layers 106. In some embodiments, tablets 420 begin dissolving essentially immediately after administration. In some embodiments, dose unit 418 cannot begin dissolving until controlled-release layers 106 dissolve. In some other embodiments, dose unit 418 slowly dissolves as body fluids penetrate controlled-release layers 106, solubilize some portion of dose unit 418, and then diffuse, at a predetermined rate, across controlled-release layers 106.

In the illustrative embodiment, one dose unit 418 is depicted in region 110, and one dose unit 420 is disposed in region 114 at each end of sleeve 102. In some other embodiments, more than one dose unit 418 is present in region 110. Likewise, as a function of the size of dose unit 420 and the size of sleeve 102 and cap 112, more than one dose unit 420 can be present at one or both of regions 114.

With reference to FIG. 5, dose units 518 and 520 are in the form of a “core.” For the purposes of this specification, a “core” is a dosage form that includes a substrate (e.g., substrate 522), a dose unit (e.g., dose unit 526) that is disposed on the substrate, and at least one cover layer (e.g., cover layer 524). The cover layer is attached to the substrate and covers the dose unit. In system 100 depicted in FIG. 5, three dose units 518 are contained in region 110, and one dose unit 520 is contained in each region 114 at the ends of sleeve 102.

Region 110 advantageously, but not necessarily, has excess or unoccupied space, even after it receives its full complement of dose units (e.g., one dose unit 418, three dose units 518, etc.). This excess space improves the flow of fluid through region 110 (once body fluids gain access to that region), thereby enhancing dissolution of the dose unit(s). If excess space is provided for the purposes of improving flow, it will be in a range of about 5 percent to 30 percent excess.

As indicated above, once the dosage forms within region 110 are exposed to body fluids, the availability of drug depends upon certain characteristics of the dosage form. Some of those characteristics are now described below.

Although most dosage forms can be adapted for immediate release or at least a slight delay on exposure to body fluids, the “core” dosage form (see, e.g., FIG. 5) is particularly useful for providing a secondary delay and for affecting the drug-delivery profile.

In some embodiments of a core dosage form, the cover layer is non-permeable and must first dissolve before drug can be released. The rate of dissolution of the cover layer can be varied as a function of the composition and/or thickness of the cover layer. In this manner, the core dosage form, and more particularly the cover layer of the core, provides a secondary control mechanism for delaying drug delivery.

In some additional embodiments, the cover layer can be insoluble but permeable, such that drug must diffuse across the cover layer for release into an animal's system. This provides a secondary control mechanism for moderating the rate of release of drug.

In some further embodiments, multiple cores are present in region 100, wherein each core has a cover layer with characteristics that are different from the cover layers of other cores. This provides substantial flexibility in moderating the rate of release of drug from drug-delivery system 100.

Varying the composition of dose units also provides an ability to tailor the drug-delivery profile of drug-delivery system 100. In the case of drug-delivery systems that incorporate multiple dose units, the dose units can all contain the same drug or drugs and at the same concentration(s), or they can contain the same drug(s) at different concentrations, or they can contain different drugs.

Design of controlled-release drug delivery system 100 will be dictated, to a large extent, by the desired plasma profile. The correlation between the desired in vivo plasma profile and an in vitro dissolution profile of the drug (in vitro, in vivo correlation (IVIVC)) can be used in design and testing of the dosage form. The in vitro dissolution profile of a delivery system made in accordance with the illustrative embodiment of the present invention can be measured by means known to those skilled in the art. The IVIVC is known for many drugs or can be determined by those skilled in the art according to known methods. Such methods are generally described, for example, in a publication published in September 1997 by Food and Drug Administration, Center for Drug Evaluation and Research, entitled “Guidance for Industry, Extended Release Oral Dosage Forms: Development, Evaluation, and Application of In Vitro/In Vivo Correlations.” Computer software is commercially available for predicting plasma profiles from orally-delivered drugs.

In many cases, the desired release profile includes an initial release of drug to achieve a base drug level, followed by extended release to substantially maintain the base level. This type of profile can be obtained using drug delivery system 100. Several parameters that should be considered in the design of the dosage form to achieve a desired release profile are:
the amount of drug needed in the immediate-release component;
the duration of the immediate release;
the amount of drug that needs to be released during controlled-release pulses of drug;
the time delay, if any, until controlled-release begins;
the desired location of drug release;
whether drug release is to be pH independent or pH dependent;
the duration of each controlled-release pulse of drug; and
the total duration of time for controlled release.

Additional parameters to be considered in the design of controlled-release drug delivery system 100 to achieve a desired release profile are:
the amount of drug per dose unit;
the number of dose units;
the material(s) used for the core's substrate and cover layer (in embodiments that use a core as the dosage form);
the material(s) used for controlled-release layers 106;
the material(s) used for sleeve 102;
the thickness and number of layers used for the substrate, the cover layer, the controlled-release layers 106, and sleeve 102; and
the manner in which the dose units are assembled.
   All of these parameters can be controlled in controlled-release drug delivery system 100 in accordance with the illustrative embodiment of the present invention.

The foregoing provides a description of the structure of controlled-release drug delivery system 100. Additional detail concerning the various elements of system 100 is now provided.

Sleeve 102 and CaD 112

Sleeve 102 is formed of an impermeable, non-flexible material that is suitable for ingestion by an animal. In some embodiments, sleeve 102 is formed of cellulose acetate or cellulose acetate butyrate. Pharmaceutical-grade cellulose acetate is available from Eastman Chemical. Tubes that are made from such cellulose acetate are available from Petro Packaging of Cranford, N.J. and others. The tubes are machined, as appropriate, to form sleeve 102.

Additionally, other materials that are non-flexible, but otherwise permeable, such as acrylate resins, can be coated with wax, such as a combination of paraffin and microcrystalline wax, or carnauba wax and beeswax, in known fashion, to render them impermeable.

Sleeve 102 must be small enough for administration. For example, for oral administration, the sleeve should have a diameter that is in a range from about 3 millimeters to about 8 millimeters, and a length that is in a range from about 3 millimeters to about 8 millimeters. Sleeve 102 advantageously (but not necessarily) has a cylindrical shape, which eases oral administration.

Cap 112 is advantageously formed of the same material as sleeve 102.

Controlled-Release Layer 106

As previously noted, the dissolution of controlled-release layers 106 is the primary mechanism for delaying the release of drug from system 100. Variables that affect the dissolution rate of controlled-release layers 106 include the composition of controlled-release layer 106, its thickness, and the design of delivery system 100 itself (e.g., how far layers 106 are recessed within sleeve 102, etc.). Likewise, diffusion through controlled-release layers 106 is a primary mechanism for creating a sustained drug release.

Controlled-release layers 106 are advantageously made of a material that has adequate mechanical stability; pharmaceutical acceptability; and non-reactivity with the drug being used. In embodiments in which it is intended that controlled-release layers 106 dissolve, it is advantageous for them to completely dissolve. Complete dissolution of each controlled-release layer promotes full dissolution of the dose unit(s).

Many different types of materials can be used for controlled-release layers 106, including, without limitation, polymers and matrix-type materials such as inorganic materials. In some embodiments, nonwoven fabrics are used. Polymers suitable for use as controlled-release layers 106 include, without limitation, polyvinylacetate, polyvinylalcohol, polyvinylpyrrolidone (PVP), polyethylene oxide (PEa), gelatin, modified starches, and celluloses such as hydroxypropylmethyl cellulose (HPMC), methyl cellulose (MC), ethyl cellulose (EC), and hydroxypropyl cellulose (HPC). In a preferred embodiment, K-laO grade HPMC, such as is available from Dow and others, is used.

Appropriate selection of the materials for the controlled-release layers 106 enables the layers to function as both a delaying mechanism and a sustained-release mechanism. For example, using an appropriate material, or combination of materials, the diffusion properties of layer 106 can be made to vary as a function of pH, or other parameters. Thus, the diffusion of drug can be kept quite low, for example, until delivery system 100 reaches the small intestine, etc.

In embodiments in which sleeve 102 has an internal diameter of about 8 millimeters or less, which is typical, controlled-release layers 106 have a thickness in a range of between about 20 microns and about 250 microns. Due to manufacturing constraints, controlled-release layers having a thickness of about 150 microns or more might be implemented as two, relatively thinner layers that together possess a desired thickness.

Dose Unit

The amount of drug per dose unit will vary depending upon the drug or drugs to be delivered and the desired plasma profile.

Many active agents can be formulated into dosage forms for use in conjunction with controlled-release drug delivery system 100. Examples include, without limitation, synthetic and isolated organic and inorganic compounds or molecules, proteins and peptides, polysaccharides and other sugars, lipids, and nucleic acid molecules. The active agents can have any of a variety of activities or functions, which may be inhibitory or stimulatory, including, without limitation, materials that act upon the central nervous system such as hypnotics, sedatives, psychic energizers, tranquilizers, antidepressants, and anticonvulsants; muscle relaxants; muscle contractants; antiparkinson agents; agents having antibiotic activity, antiviral activity, antifungal activity, steroidal activity, cytotoxic or anti-proliferative activity, anti-inflammatory activity, analgesic or anesthetic activity, anti-HIV agents, antiemetics, pain relievers, hormones, antiangiogenic agents, antibodies, neurotransmitters, psychoactive drugs, drugs affecting reproductive organs, and oligonucleotides such as antisense oligonucleotides, as well as contrast or other diagnostic agents. A description of these classes of drugs and listing of species within each class can be found in Martindale, The Extra Pharmacopoeia, 31 st Ed., The Pharmaceutical Press, London (1996) and Goodman and Gilman, The Pharmacological Basis of Therapeutics, (9th Ed., McGraw-Hill Publishing Company (1996).

The amount of drug that will be incorporated in a dose unit varies widely depending on the particular drug, the desired therapeutic effect, and the time span necessary for the drug to be released. A variety of dose units in a variety of sizes, shapes and compositions are intended to provide complete dosage regimes for therapy for a variety of maladies. Thus, it is not practical to define a range for the therapeutically-effective amount of drug to be ref eased by the individual units or by the controlled-release drug delivery system as a whole.

In some cases it is advantageous to add excipients to the drug. Excipients, whije not bioactive, provide a functionality, which typically relates to a physical process. Excipients that are suitable for combining with pure drug include, without limitation, diluents, disintegrants, dispersants, preservatives, stabilizers, thickeners, and combinations of any two or more thereof. Excipients that perform multiple functions can also be included in the composition.

In cases in which the drug being used is a basic drug, it is often desirable to add an acid excipient, such as citric acid, to the dose unit. Acid excipients can accelerate dissolution, which is useful for the following reason. It is known that the solubility of a basic drug decreases as pH increases. So, to compensate for the decrease in solubility that would otherwise occur as drug moves from the stomach to the small intestine for example, an acid excipient is advantageously used.

Dosage Form

In some embodiments, a dose unit includes drug in solid form (e.g., a tablet, caplet, etc.). In some other embodiments, the drug is present in a powdered form. In some embodiments in which the drug is present in a powdered form, the drug is contained within a package (e.g., capsule, as part of a core between a cover layer and base substrate, etc.). In some embodiments in which the drug is present in a powdered form, the powder is loose within sleeve 102 (in region 110 between controlled-release layers 106. In other embodiments, a dose unit can be provided in another form, such as, without limitation, a liquid, gel, or oil, as long as the liquid, oil, or gel does not detrimentally interfere with the dissolution (and/or diffusion) properties of controlled-release layers 106.

In some embodiments, the dose units are provided as deposits (previously defined). A deposit is made by a method that suitably applies a controlled amount of a drug onto a substrate. One method for doing this is to electrostatically deposit a dosage amount of drug onto an appropriate substrate.

In some electrostatic deposition processes, a cloud or stream of charged particles of drug is directed towards an oppositely-charged substrate. A measured

dosage of the drug, in particulate form, deposits on the substrate without the need for additional carriers, binders or the like. Electrostatic deposition can form a stable layer of a drug, with or without excipients, which would otherwise be unstable.

In some embodiments, the deposition is sealed in place on the deposition substrate by attaching a cover layer, creating a “core,” as previously described. For example, the cover can be bonded to the substrate around the perimeter of the deposited drug. This entraps the powdered drug between the substrate and cover layers, forming a laminate. Electrostatic deposition techniques are described, for example, in U.S. Pat. Nos. 5,753,302, 5,788,814, 5,858,099, 5,846,595 to Sun et al., U.S. Pat. No. 5,871,010 to Datta et al., U.S. Pat. Nos. 5,669,973 and 5,714,007 to Pletcher et al., and PCT/US99/12772 by Chen et al, filed on Jun. 8, 1999. These and other known methods and apparatuses for deposition and formation of laminates can suitably be used. All these patents and patent applications are incorporated by reference herein.

Material suitable for use as a substrate for electrostatic deposition possesses the following general characteristics: consistent electrical properties; adequate mechanical stability; optical properties suitable for dose measurement. In some embodiments, substrate-suitable materials exhibit one or more of the following additional characteristics: is suitable for lamination, possesses pharmaceutical acceptability; and is non-reactive with the drug powder(s). Illustrative materials suitable for use as a substrate for an electrostatic deposition process include, without limitation, polymers, non-woven fabrics, paper, inorganic materials such as metal salts and metal alloys, and cellulose materials.

The deposition substrate advantageously comprises a polymeric substance that dissolves in body fluids. In an alternative embodiment, the substrate is an indestructible substance that is readily eliminated from the body once the drug has been released from the dose unit into the body. Polymers suitable for use as a deposition substrate include, without limitation, polyvinylacetate, polyvinylalcohol, polyvinylpyrrolidone (PVP), polyethylene oxide (PEa), gelatin, modified starches, and celluloses such as hydroxypropylmethyl cellulose (HPMC), methyl cellulose (MC), ethyl cellulose (EC), and hydroxypropyl cellulose (HPC).

The polymeric substance for use as a deposition substrate is advantageously available as a film. In some embodiments, the film includes a plasticizer

to increase the flexibility of the film. A suitable plasticizer is polyethylene glycol (PEG); other plasticizers suitable for use are known to those skilled in the art.

The substrate can have any thickness so long as it functions as described above. In general, the thickness will be between about 0.0002 and 0.02 inches, desirably about 0.001 inches (0.0254 mm).

Release of the drug from the substrate can be immediate, upon exposure to an environment in which the substrate or the drug is soluble, such as gastric fluid. Alternatively, drug release can be dependent, to varying degrees, upon dissolution of the substrate in the environment. Accordingly, in some embodiments, the deposition substrate is a factor in the overall release profile, while in other embodiments, it has an insignificant effect.

As described above, a cover layer can be used to form a laminate comprising the substrate, the dose unit, and the cover. Use of a cover is not necessary but can be advantageous to provide structural integrity to the deposits. The cover need not have the same electrical properties as the substrate, but should exhibit adequate mechanical stability; properties suitable for lamination; pharmaceutical acceptability; and non-reactivity with the drug powder(s).

As previously noted, in some embodiments, the cover layer is a factor in the overall release profile of the dosage form, while in other embodiments, it has an insignificant effect. To that end, in some embodiments, a cover film can be used that provides modified release of the drug. Immediate release can be provided by a cover layer that is made of a material that dissolves very quickly. Delayed release of the drug can be provided by use of a cover that has delayed dissolution in the environment. And sustained release can be provided through use of a cover layer that provides controlled transport of the drug. For example, the cover layer can be made of a material that forms a gel upon contact with gastric fluid.

In some embodiments, the cover layer comprise a polymeric film such as, without limitation, polyvinylacetate, polyvinylalcohol, polyvinylpyrrolidone (PVP), polyethylene oxide (PEO), gelatin, modified starches, and celluloses such as hydroxypropylmethyl cellulose (HPMC), methyl cellulose (MC), ethyl cellulose (EC), and hydroxypropyl cellulose (HPC). It is within the capabilities of those skilled in the art to select a material to provide the desired release characteristic. The cover can be the same material as the substrate or it can be a different material.

The cover layer can have any thickness so long as it functions as described above. In general, the thickness will be between about 0.0002 and 0.2 inches, desirably about 0.001 inches (0.0254 mm), consistent with substrate thickness.

The drug-delivery system described herein is useful for controlled delivery of a variety of drugs. While the illustrative system is described in the context of oral administration, it is not limited thereto. The same system can generally be used, for example, as vaginal and rectal suppositories. In the case of these other forms, as well as implants, the controlled-release layers are advantageously formed from more slowly-dissolving materials, since the time of residence of the drug-delivery system will be substantially longer than the about 24-hour residence time of oral dosage forms. Indeed, drug delivery from implants takes place over the course of weeks or months. To achieve such protracted erosion, biodegradable polymers are advantageously used. These are insoluble polymers that gradually break down in vivo. Examples include, without limitation, polyorthoesters, lactide and glycolide polymers and copolymers, polyesteramides, and hydroxybutyrate-hydroxyvalerate co-polymers.

In some embodiments, drug-delivery system 100 includes a dissolution-enhancing amount of a surfactant. The surfactant can be:
incorporated into/onto controlled-release layers 106; or
incorporated into/onto the deposition substrate (e.g., substrate 522); or
incorporated into/onto the cover layer (e.g., cover layer 524)
an independent layer (not depicted); or
incorporated into two or more of the above elements.

As the surfactant-containing element dissolves, surfactant is released in the immediate vicinity of a drug that has likewise been released from one or more dose units. The surfactant improves the dissolution, and, as a consequence, the bioavailability of low-solubility drugs (e.g., hydrophobic drugs, etc.). The use of surfactants to improve dissolution of hydrophobic drugs is disclosed in U.S. patent application Ser. No. 09/925,348, entitled “Improved Solid Pharmaceutical Dosage Formulation of Hydrophobic Drugs,” incorporated by reference herein.

In some further embodiments, a variety of other types of pharmaceutical additives (instead of or in addition to a surfactant) are advantageously incorporated into/onto controlled-release layers 106, and/or the deposition substrate (e.g., substrate 522), and/or the cover layer (e.g., cover layer 524). Pharmaceutically acceptable additives include, without limitation, antioxidants, antimicrobial agents, complexing agents, acidity-boosting agents, alkalinity-boosting agents, buffering agents, carrier molecules, chelating compounds, preservatives and the like. The use of such additives is described in further detail in Ser. No. 09/925,348, referenced above.

For oral administration, drug delivery system 100 with a plurality of dose units between controlled-release layers 106, are advantageously designed for release of drug at a frequency ranging from release once about every hour to release once about every 12 hours. Preferably, release of drug occurs once about every 2 hours to once about every 6 hours. Since the maximum time that a typical oral dosage form will remain in the body is about 24 hours, a drug-delivery system in accordance with the illustrative embodiment will contain from about 2 to about 24 dose units, more typically about 4 to 12 dose units. Of course, these numbers will vary when the system is intended for applications in which it will be retained in an animal for longer than 24 hours.

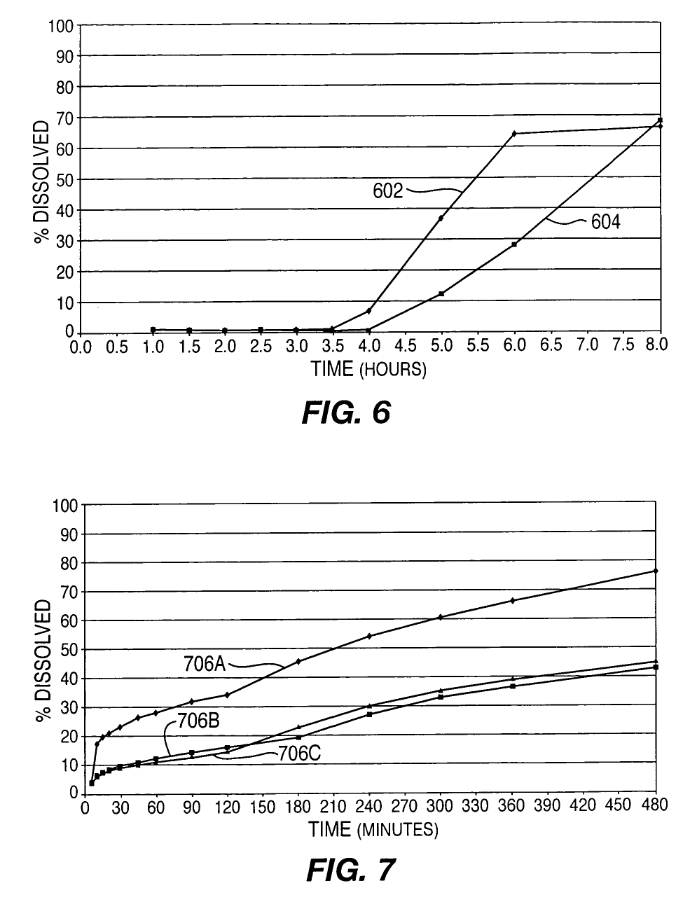

Further description of drug delivery system 100 is provided by way of Examples I through VII and FIGS. 6 through 15. The Figures show release curves, which are based on experimental data that was obtained using variations of drug delivery system 100. It is to be understood that these Examples are provided by way of illustration, not limitation.

The data reported that is reported below was obtained using a U.S. Pharmacopeia “Apparatus 2,” at 50 rpm, with JP Sinker cages. The test medium was either simulated intestinal fluid (pH 6.8), 0.01N hydrochloric acid, intestinal fluid (without enzymes), or gastric fluid (without enzymes).

Drug delivery system 100 included a cylindrical sleeve (3 to 6 millimeters diameter), two end caps, and two controlled-release layers. The sleeve and end caps were formed of cellulose acetate unless otherwise noted. The controlled-release layers were hydroxypropylmethyl cellulose (K-100 LV available from DOW) unless otherwise noted, and varied in thickness (c.a., 30-250 microns).

Located within the sleeve between the controlled-release layers were one or more dose units of active ingredient. Dose units that were disposed between the controlled-release layers served as controlled-release components. For some of the experiments, one or more dose units of active ingredient were disposed near one or both ends of the sleeve, on the (medium-) exposed side of the controlled-release layers. These “exposed” dose units served as immediate-release components. The terms “controlled-release” and “immediate-release,” as used in the Examples below, refer to the location of a dose unit relative to the controlled-release layers (and, consequently, its accessibility to the ambient environment), consistent with the foregoing description.

The active ingredient was in the form of a tablet, core or loose powder. The tablet included an amount of active ingredient, and varying proportions of excipients, including citric acid, lactose, Prosolve SMCC 90, Explotab, and magnesium stearate. The cores and powder contained pure active ingredient or a combination of active ingredient and pH modifier.

### EXAMPLE I

### Delayed Release

This Example provides an illustration of delayed drug release from drug delivery system 100 into simulated intestinal fluid. Results are shown for two lots of tablets, both containing 12.5 milligrams of active ingredient but varying in the proportions of the excipients listed above. The controlled-release layers had a thickness of about 50 microns. For this Example, drug delivery system 100 was configured with a single tablet between the controlled release layers and no immediate-release components. Plots 602 and 604 in FIG. 6 show that drug delivery is delayed 3.5 hours and 4 hours after “administration,” respectively, for the two lots of tablets.

### EXAMPLE II

### Sustained Release

This Example provides an illustration of sustained release from drug delivery system 100. Results are shown for two variations of drug delivery system 100. In one variation, drug delivery system 100 includes two tablets each containing 5 milligrams of active ingredient. One of the tablets served as an immediate-release component (i.e., it was on the exposed side of the controlled-release layers) and the second tablet served as a controlled-release component (i.e., it was disposed between the controlled-release layers). The controlled-release layers had a thickness of about 250 microns and the medium was 0.01 N hydrochloric acid.

Plots 706A, 706B, and 706C in FIG. 7 depict results for three samples of this variation of system 100. The effects of the immediate and controlled-release components can be seen. In particular, plots 706A, 706B, and 706C shows an initial relatively rapid release of active ingredient (i.e., in the first fifteen to thirty minutes) and then gradual release over the next seven and one-half hour period.

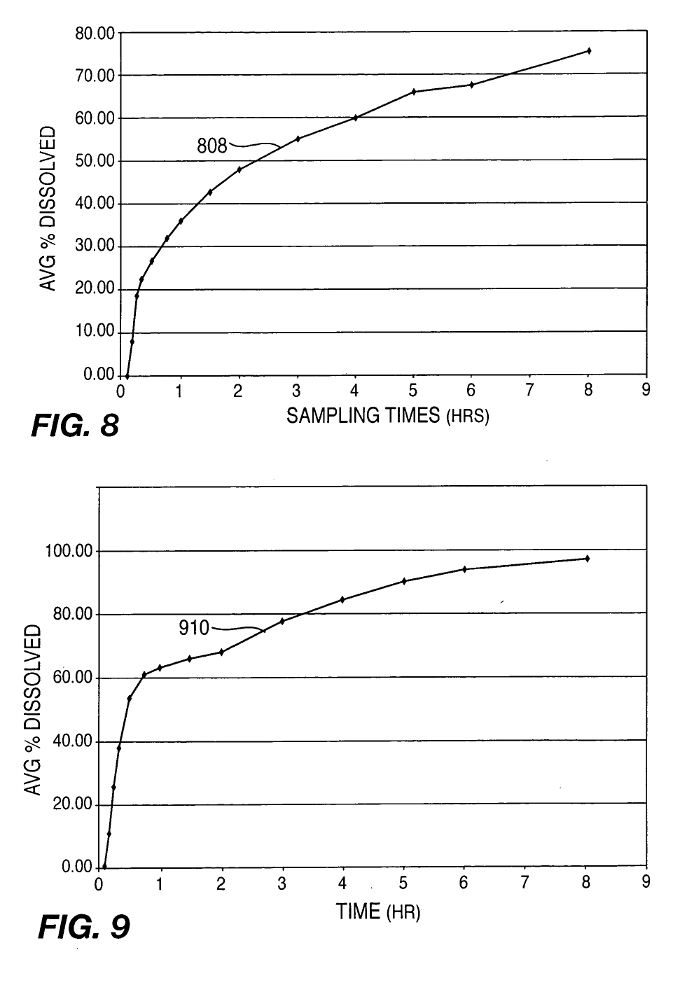

In a second variation, drug delivery system 100 includes three, 4-milligram cores. One of the cores served as a controlled-release component and the other two were exposed for immediate release. This variation was tested separately in intestinal fluid and gastric fluid. Plot 808 in FIG. 8 depicts the averaged results for six samples of this variation of system 100 in intestinal fluid. Plot 808 shows sustained release of active ingredient, beginning with an initial rapid release of active ingredient for the first fifteen minutes, and continuing release at a decreasing rate over the next seven hours and forty-five minutes. Plot 910 in FIG. 9 depicts the averaged results for six samples of this variation of system 100 in gastric fluid. Plot 910 shows that compared with intestinal fluid, a greater percentage of active ingredient was released in gastric fluid during the initial rapid release period (i.e., 55 percent dissolved vs. 27 percent dissolved in 30 minutes). Ultimately, a greater amount of active ingredient was released over time in the gastric fluid as compared to the intestinal fluid.

### EXAMPLE III

### Immediat Release, then Delay, then Sustained Release

This Example illustrates a release profile that is characterized by immediate release, then a period of delay, and a period of sustained release of active ingredient following the delay. Results are shown for release into simulated intestinal fluid. For this Example, drug delivery system 100 included one immediate-release tablet and one controlled-release tablet, both containing 5 milligrams of active ingredient.

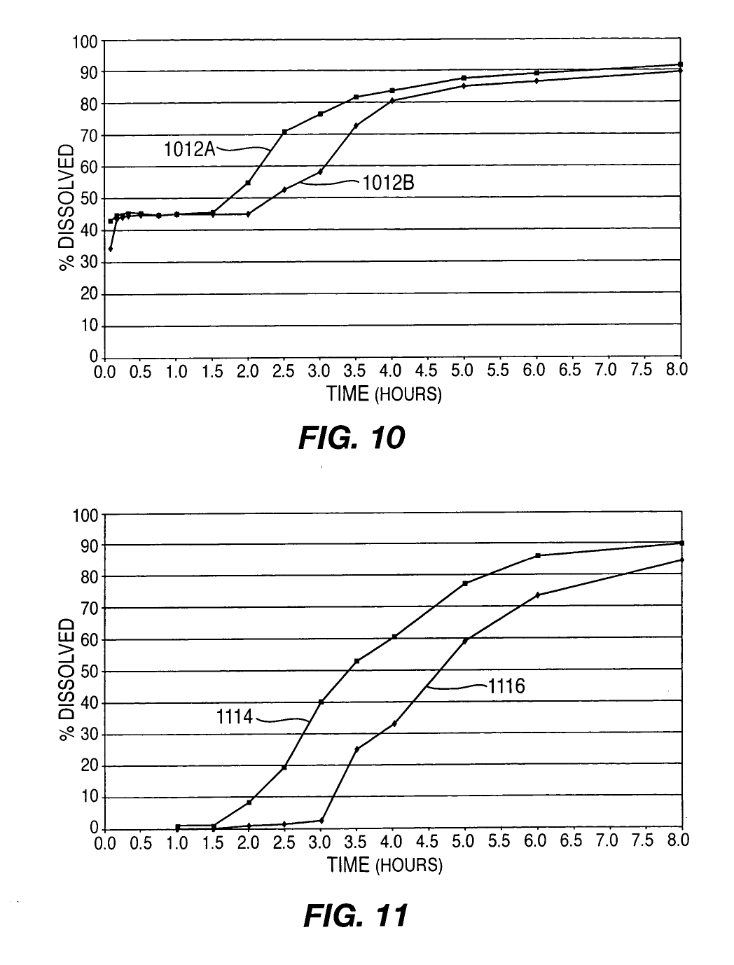

Plots 1012A and 1012B in FIG. 10 depict the release profile for two lots of active ingredient into simulated intestinal fluid. The release profiles show that in the first few minutes, most of the immediate release tablet is dissolved. For the next hour and twenty minutes, no further active ingredient is released. After this period of delay, a period of sustained release begins and continues for the next 6 hours and thirty minutes.

### EXAMPLE IV

### Variation in Composition of the Controlled-Release Layer

This Example provides an illustration of a manner in which a drug-release profile can be affected by varying the composition of the controlled-release layer. In particular, a first set of runs was conducted using controlled-release layers that were 90 percent HPMC and 10 percent “Kollicoat IR,” a sustained-release formulation available from BASF of Ledgewood, N.J. (typically, polyvinyl alcohol polyethylene glycol graft copolymer). A second set of runs was conducted in which the controlled-release layers were 75 percent HPMC and 25 percent Kollicoat IR. For all runs, drug delivery system 100 included a single, 5-milligram, controlled-release tablet. The medium was simulated intestinal fluid.

Plot 1114 in FIG. 11 shows the release profile for drug delivery system 100 having controlled release layers of 10 percent Kollicoat IR (average of six runs). Plot 1116 shows the release profile for drug delivery system 100 having controlled-release layers of 25 percent Kollicoat IR (average of three runs). As shown by plots 1114 and 1116, the controlled-release layers having the greater proportion of Kollicoat (plot 1116) provided a longer period of delay before release of active ingredient.

### EXAMPLE V

### Variation in Thickness of the Controlled-Release Layer

This Example provides an illustration of a manner in which the drug-release profile can be affected by varying the thickness of the controlled-release layers. A first set of runs was conducted using controlled-release layers that had a thickness of about 31-32 microns. A second set of runs was conducted using controlled-release layers that had a thickness of about 40-42 microns. For all runs, two immediate-release tablets having 7.5 milligrams of active ingredient and one controlled-release tablet having 5.0 milligrams of active ingredient were used. The medium was simulated intestinal fluid.

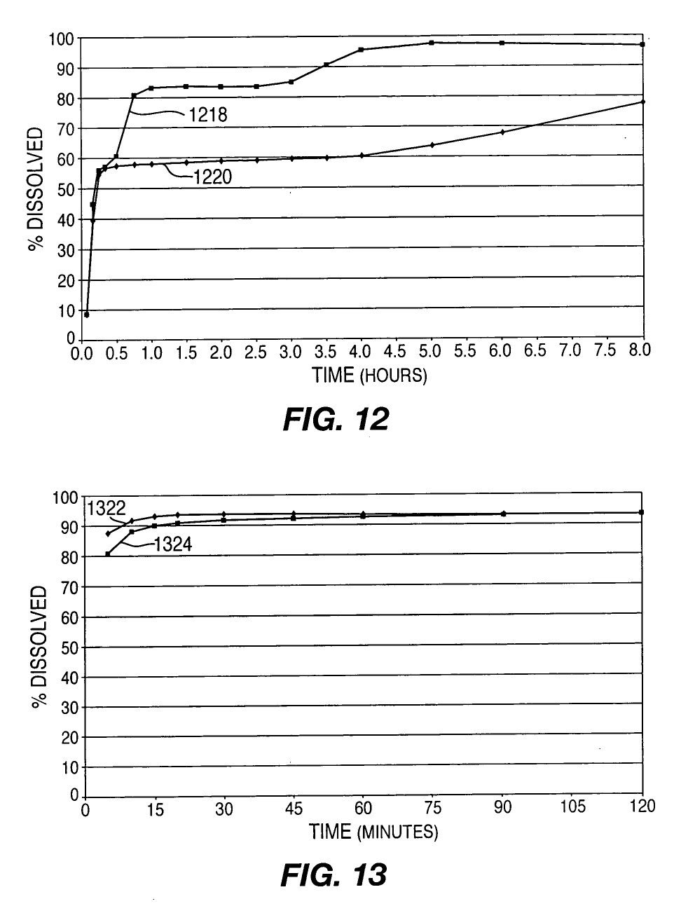

Plot 1218 in FIG. 12 depicts the release profile for drug delivery system 100 that included controlled-release layers having a thickness of about 31-32 microns (average of three runs). Plot 1220 in FIG. 12 depicts the release profile for drug delivery system 100 that included controlled-release layers having a thickness of about and 40-42 microns (average of three runs). The plots shows that, after the period of immediate release, drug delivery system 100 having the relatively thicker controlled-release layers (plot 1220) caused a greater delay in the release of active ingredient. After eight hours, about twenty-percent less active ingredient was released from the system having the relatively thicker controlled-release layers.

### Example VI

### Affect of Excipients

This Example provides an illustration of a manner in which the drug-release profile can be affected by varying the excipient composition of a dose unit (e.g., tablet, etc.). For these runs, drug delivery system 100 included an immediate-release tablet having 5 milligrams of active ingredient. Drug delivery system 100 did not include a controlled-release tablet.

In this case, the active ingredient is basic. It is often desirable to add an acid excipient, such as citric acid, to a basic active ingredient. In particular, acid excipients can accelerate dissolution, and it is known that the solubility of a basic drug decreases as pH increases. So, to compensate for the decrease in solubility that would otherwise occur as active ingredient moves from the stomach to the small intestine for example, an acid excipient is advantageously used.

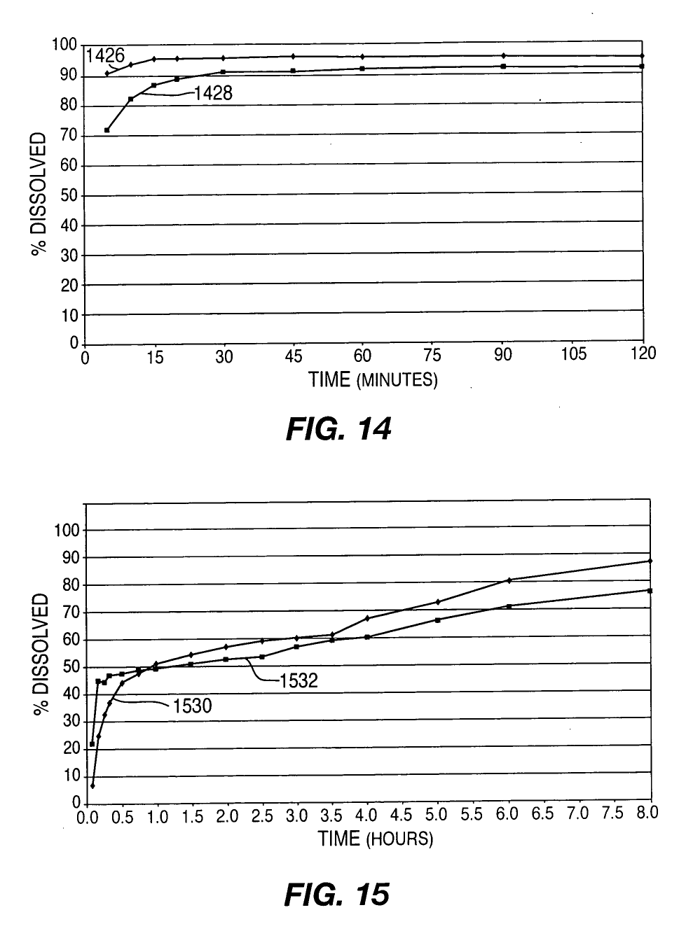

FIG. 13 depicts release profiles (average of six runs) for drug delivery system 100 having an immediate-release tablet that contained 5 percent citric acid. Plot 1322 in FIG. 13 shows the release profile in an acid medium (0.01N hydrochloric acid) and plot 1324 shows the release profile in a basic medium (simulated intestinal fluid pH 6.8). FIG. 14 depicts the release profile (average of six runs) for drug delivery system 100 having a tablet that contained no citric acid. Plot 1426 in FIG. shows the release profile in the acid medium and plot 1428 shows the release profile in the basic medium.

With reference to FIG. 13, the release profiles show that initially, for the tablet containing 5 percent citric acid, the amount of active ingredient that dissolved into the simulated intestinal fluid (plot 1324) was about 81.5 percent compared to about 88 percent into the acid (plot 1322). Within about 90 minutes, an equal amount of active ingredient had dissolved into the two mediums.

With reference to FIG. 14, which shows release profiles for the tablet that did not contain any citric acid, the amount of active ingredient that dissolved into the simulated intestinal fluid (plot 1428) was about 71.6 percent compared to about 90.6 percent into the acid (plot 1426). After 90 minutes, the amount of active ingredient that dissolved into the simulated intestinal fluid remained several percent less than the amount that dissolved into the acid.

### EXAMPLE VII

### Variation in the Form of the Dose Unit

In previous Examples, dose units were in the form of a tablet or a core. In this Example, drug delivery system 100 includes dose units that have the form of either a core or loose powder. For both types of dose units, drug delivery system 100 included two immediate-release components containing 7.5 milligrams of active ingredient and one controlled-release component containing 5 milligrams of active ingredient. The controlled-release layers had a thickness of 150-160 microns and the medium was simulated intestinal fluid.

FIG. 15, which shows release profiles for cores (plot 1530) and powder (plot 1532), shows that performance is similar for the two dosage forms. It is understood that in other variations, the release profiles could be altered to be dissimilar as desired, based on the use of particular excipients and the choice of cover layer for the core.

It is to be understood that the above-described embodiments are merely illustrative of the present invention and that many variations thereof can be devised by those skilled in the art without departing from the scope of the invention. It is therefore intended that such variations be included within the scope of the following claims and their equivalents.

1. An article comprising a controlled-release drug delivery system, said system comprising:
an inflexible sleeve, wherein said inflexible sleeve has a first end and a second end and is open at both said first and second ends; and
a first controlled-release layer and a second controlled-release layer, wherein said controlled-release layers:
are disposed within said sleeve;
are spaced-apart from one another, defining a drug-retaining region in the space between said controlled-release layers; and
admit, in a controlled manner, body fluid into said drug-retaining region.

2. The article of claim 1 wherein said controlled-release layers undergo dissolution by exposure to said body fluid, the rate of said dissolution effecting a delay of drug release into an ambient environment.

3. The article of claim 1 wherein said controlled-release layers control a rate of diffusion of body fluid into and out of said drug-retaining region.

4. The article of claim 1 further comprising at least one dose unit of drug, said at least one dose unit being disposed in said drug-retaining region.

5. The article of claim 1 wherein an inner surface of said sleeve has a first sealing surface near said first end thereof and a second sealing surface near said second end thereof, a marginal region of said first controlled-release layer abutting said first sealing surface and a marginal region of said second controlled-release layer abutting said second sealing surface.

6. The article of claim 5 further comprising:
a first cap, wherein said first cap has an open center, and wherein said first cap is received by said first end of said sleeve and abuts said marginal region of said first controlled-release layer; and
a second cap, wherein said second cap has an open center, and wherein said second cap is received by said second end of said sleeve and abuts said marginal region of said second controlled-release layer.

7. The article of claim 6 further comprising at least one dose unit of drug, said at least one dose unit being disposed in said drug-retaining region.

8. The article of claim 7 further comprising:
a dose unit of drug, disposed adjacent said first end of said sleeve outside of said drug-retaining region; and
a dose unit of a drug, disposed adjacent said second end of said sleeve outside of said drug-retaining region.

9. The article of claim 8 wherein drug(s) contained in the dose unit(s) disposed inside said drug-retaining region differ(s) from the drug of at least one of said dose units disposed outside said drug-retaining region.

10. The article of claim 8 wherein the drug of each dose unit disposed outside said drug-retaining region is different from one another.

11. The article of claim 7, wherein said dose unit of drug has a dosage form selected form the group consisting of tablet, capsule, liquid, loose powder, and a core.

12. The article of claim 7, wherein the dosage form of said dose unit(s) of drug disposed in said drug retaining region is a core.

13. The article of claim 12, wherein said core comprises a cover layer, and said cover layer affects a rate at which the drug of the dose unit(s) disposed inside said drug-retaining region is delivered from said controlled-release drug delivery system.

14. The article of claim 7, wherein the dosage form of said dose unit(s) of drug disposed in said drug-retaining region is a tablet.

15. The article of claim 1, wherein said sleeve comprises a material selected from the group consisting of cellulose acetate and cellulose acetate butyrate.

16. A method comprising:
disposing a first dose unit of a first drug in an inflexible sleeve;
disposing a first controlled-release layer and a second controlled-release layer within said sleeve on opposite sides of said first dose unit;
inserting a first cap in said sleeve to abut said first controlled-release layer, wherein said first cap has an opening; and
inserting a second cap in said sleeve to abut said second controlled-release layer, wherein said second cap has an opening.

17. The method of claim 16 further comprising disposing a second dose unit of a second drug in said sleeve within said opening of said first cap.

18. The method of claim 16 further comprising:
disposing a second dose unit of a second drug in said sleeve; and
disposing said second dose unit between said first controlled-release layer and said second controlled-release layer.
